(19) United States
(12) Patent Application Publication
(10) Pub. No.: US 2002/0007164 A1
(43) Pub. Date: Jan. 17, 2002

(54) Garment having gasket with integrated zone of elastic tension and/or stretch

(76) Inventors: Lavada Campbell Boggs, Marietta, GA (US); Hannong Rhim, Roswell, GA (US); Leslie Dobbins, Marietta, GA (US); S. Scott Friderich, Alpharetta, GA (US)

Correspondence Address: Pauley Petersen Kinne & Fejer, Suite 365, 2800 West Higgins Road, Hoffman Estates, IL, 60195, US

(21) Appl. No.: 09/855,180

(22) Filed: May 14, 2001

Related U.S. Application Data
(60) Provisional application No. 60/204,368, filed on May 15, 2000.

(51) Int. Cl. A61F 13/15; A61F 13/20

(52) U.S. Cl. 604/367; 604/385.27

(57) ABSTRACT

A disposable garment includes a gasket around one or more openings for the legs, arms, waist or the like on a wearer. At least a portion of the garment includes a targeted elastic material including zones of high and low elastic tension and/or high and low stretch in the same material, integrated during formation of the material. The targeted elastic material is positioned so that at least one high tension and/or low stretch zone is aligned with at least one garment opening, and at least one low tension and/or high stretch zone is aligned inward of the high tension and/or low stretch zone, thereby functioning as a gasket without requiring a separately manufactured, attached elastic band.

### FIELD OF THE INVENTION

This invention relates to a garment having a gasket formed from at least one integrated zone of elastic tension and/or elastic stretch aligned with a garment opening, for instance a waist opening or a leg opening.

### BACKGROUND OF THE INVENTION

Garments, including pant-like absorbent garments, medical garments, and other products, are commonly made with an elastic band adjacent at least one of the garment openings. A pant-like garment, for instance, may have an elastic band adjacent the waist opening, each of the two leg openings, or all three of the openings. The elastic bands are intended to fit snugly around a wearer's body to serve as gaskets, which prevent or reduce leakage of waste materials from inside the garment. Elastic bands have also been employed in leg flaps that provide further leakage protection in pant-like garments, and in other auxiliary gasketing applications.

In conventional garments, the primary material for the garment is manufactured and assembled separately from the elastic bands. Following their separate manufacture, the elastic bands are attached to the primary material at some stage during manufacture of the garment by sewing, ultrasonic welding, thermal bonding, adhesive bonding, or the like. In the resulting product, the user can often see the elastic band as a distinct entity attached to the garment.

Because of competition, there is an incentive to reduce both material and manufacturing costs associated with garments, without sacrificing performance and quality. However, this should be accomplished without compromising the gasketing characteristics around the openings in the garment. Conventional elastic bands can be relatively expensive to incorporate into garments, because of the current need for separate manufacture and attachment of the bands.

### SUMMARY OF THE INVENTION

The present invention is directed to a garment having one or more garment openings for the wearer's waist, legs, arms, and the like. The garment has elasticized gasket-like openings that provide superior leakage protection against matter either leaving or entering the garment. Furthermore, the garment can be produced without the use of a separately manufactured, separately attached elastic band, and is easier and less expensive to manufacture than a conventional garment having one or more elastic bands at the opening.

The garment of the invention is manufactured using a targeted elastic material (“TEM”) having a targeted elastic zone aligned with the garment opening or openings. The TEM may have a substantially homogeneous appearance, and does not have a separately manufactured elastic band attached to it. Yet the TEM has different elastic properties at different regions, and exhibits greater elastic tension and/or greater elongation in a region aligned with, and in the vicinity of, at least one garment opening. The TEM may also include a barrier film that is liquid impermeable and gas permeable, as a further measure of sealing fluid within the garment while allowing moisture vapor to escape.

With the foregoing in mind, it is a feature and advantage of the invention to provide a garment having a targeted gasket-like elastic region aligned with, and in the vicinity of at least one garment opening, while eliminating the separate manufacture and attachment of an elastic band.

It is also a feature and advantage of the invention to provide various techniques for providing a garment with a targeted elastic material having a targeted gasket-like elastic region aligned with, and in the vicinity of, at least one garment opening.

These and other features and advantages will become further apparent from the following detailed description of the presently preferred embodiments, read in conjunction with the drawings.

### BRIEF DESCRIPTION OF THE DRAWINGS

FIG. 1 illustrates a perspective view of a pant-like absorbent garment in accordance with the invention, having targeted elastic gasket regions aligned with, and in the vicinity of garment openings;

FIG. 2 is a plan view of the garment shown in FIG. 1, showing the side facing away from the wearer;

FIG. 3 is a plan view of the garment shown in FIG. 1, showing the side facing the wearer;

FIGS. 4-7 illustrate representative targeted elastic laminate (“TEL”) materials useful for making the garments of the invention;

FIGS. 8-11 illustrate representative processes for making TEL materials useful for making garments of the invention;

FIG. 12A shows one exemplary adhesive spray pattern in which the adhesive has been applied to the elastic filaments with attenuation in the cross direction;

FIG. 12B shows a second exemplary adhesive spray pattern;

FIG. 12C illustrates a third exemplary adhesive spray pattern;

FIG. 12D shows an exemplary bond angle in one exemplary adhesive spray pattern;

FIG. 13 illustrates the bonding pattern and method of calculating the number of bonds per unit length on elastic strands or filaments;

FIG. 14A shows a fourth exemplary adhesive spray pattern in a swirled-type of configuration;

FIG. 14B shows a fifth exemplary adhesive spray pattern that is more randomized and which provides a large percentage of adhesive lines in a perpendicular orientation to the elastic filaments;

FIG. 14C illustrates a sixth exemplary adhesive spray pattern having attenuation of adhesive lines in the cross-machine direction;

FIG. 14D shows a seventh exemplary adhesive spray pattern that resembles a “chain-link fence”; and

FIG. 15 is a schematic view of another process for making TEL materials useful for making garments of the invention.

### DEFINITIONS

The term “elastic band” refers to a discrete elongated element having elastic properties. The term “discrete elongated element” refers to a long, relatively narrow element that is separately manufactured and then attached to an underlying material, and does not include elongated regions having elastic properties that are part of an underlying material as made. The terms “elastic” and “elastomeric” are used interchangeably to mean a material that is generally capable of recovering its shape after deformation when the deforming force is removed. Specifically, as used herein, elastic or elastomeric is meant to be that property of any material which upon application of a biasing force, permits that material to be stretchable to a stretched biased length which is at least about 50 percent greater than its relaxed unbiased length, and that will cause the material to recover at least 40 percent of its elongation upon release of the stretching force. A hypothetical example which would satisfy this definition of an elastomeric material would be a one (1) inch sample of a material which is elongatable to at least 1.50 inches and which, upon being elongated to 1.50 inches and released, will recover to a length of not more than 1.30 inches. Many elastic materials may be stretched by much more than 50 percent of their relaxed length, and many of these will recover to substantially their original relaxed length upon release of the stretching force.

The term “inelastic” refers to materials that are not elastic.

The term “gasket” or “gasket region” refers to a region of a garment which exhibits a moderate level of elastic tension against a wearer's body during use, and which restricts the flow of liquid and other material through a garment opening between the inside and outside of the garment. The term “fluid sealing gasket” is synonymous with these terms.

The term “targeted elastic regions” refers to isolated, often relatively narrow regions or zones in a single composite material or layer, which have greater elastic tension and/or elongation than adjacent or surrounding regions.

The term “targeted elastic material” (“TEM”) refers to a single elastic material or laminate having targeted elastic regions. TEM's include only materials or laminates which are made in a single manufacturing process, and which are capable of exhibiting targeted elastic properties without requiring an added elastic band or layer in the targeted elastic region. TEM's do not include materials having elasticized regions achieved through separate manufacture of an elastic band, and subsequent connection of the elastic band to the underlying material.

The term “targeted elastic laminate” or “TEL” refers to an elastic laminate which behaves as a TEM. The TEL suitably includes at least one elastic nonwoven filament web, in which different zones of different elastic tension and/or elongation exist across a width of the web when the laminate is stretched in a longitudinal direction perpendicular to the width. What is important is that the different zones exhibit different levels of retractive force when the laminate is uniformly stretched by a selected amount. The elastic nonwoven filament web is laminated to at least one other layer, whereby the laminate exhibits different levels of elastic tension and/or elongation in zones corresponding to the high and low tension zones in the nonwoven filament web.

The term “targeted elastic stretch-bonded laminate” or “TE SBL” refers to a TEL which is formed by stretching the elastic nonwoven filament web having the zones of different elastic tension and/or elongation, maintaining the stretched condition of the elastic nonwoven filament web when the other layer is bonded to it, and relaxing the TEL after bonding.

The term “vertical filament stretch-bonded laminate” or “VF SBL” refers to a stretch-bonded laminate made using a continuous vertical filament process, as described herein.

The term “continuous filament stretch-bonded laminate” or “CF SBL” refers to a stretch-bonded laminate made using a continuous horizontal filament process, as described herein.

The term “elastic tension” refers to the amount of force per unit width required to stretch an elastic material (or a selected zone thereof) to a given percent elongation.

The term “elongation” refers to the capability of an elastic material to be stretched a certain distance, such that greater elongation refers to an elastic material capable of being stretched a greater distance than an elastic material having lower elongation.

The term “low tension zone” or “lower tension zone” refers to a zone or region in a stretch-bonded laminate material having one or more filaments with low elastic tension characteristics relative to the filament(s) of a high tension zone, when a stretching or biasing force is applied to the stretch-bonded laminate material. Thus, when a biasing force is applied to the material, the low tension zone will stretch more easily than the high tension zone. At 50% elongation of the fabric, the high tension zone may exhibit elastic tension at least 10% greater, suitably at least 50% greater, desirably about 100-800% greater, alternatively about 150-300% greater than the low tension zone.

The term “high tension zone” or “higher tension zone” refers to a zone or region in a stretch-bonded laminate material having one or more filaments with high elastic tension characteristics relative to the filament(s) of a low tension zone, when a stretching or biasing force is applied to the stretch-bonded laminate material. Thus, when a biasing force is applied to the material, the high tension zone will stretch less easily than the low tension zone. Thus, high tension zones have a higher tension than low tension zones. The terms “high tension zone” and “low tension zone” are relative, and the material may have multiple zones of different tensions.

The term “low stretch zone” or “lower stretch zone” refers to a zone or region in a stretch-bonded laminate material having one or more filaments with low elongation characteristics relative to the filament(s) of a high stretch zone, when a stretching or biasing force is applied to the stretch-bonded laminate material. Thus, when a biasing force is applied to the material, the low stretch zone will not stretch as great a distance as the high stretch zone.

The term “high stretch zone” or “higher stretch zone” refers to a zone or region in a stretch-bonded laminate material having one or more filaments with high elongation characteristics relative to the filament(s) of a low stretch zone, when a stretching or biasing force is applied to the stretch-bonded laminate material. Thus, when a biasing force is applied to the material, the high stretch zone will stretch a greater distance than the low stretch zone.

The term “nonwoven fabric or web” means a web having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The term also includes films that have been slit into narrow strips, perforated or otherwise treated to allow air to pass through. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The term “microfibers” means small diameter fibers having an average diameter not greater than about 75 microns, for example, having an average diameter of from about 1 micron to about 50 microns, or more particularly, having an average diameter of from about 1 micron to about 30 microns.

The term “spunbonded fibers” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Petersen, and U.S. Pat. No. 3,542,615 to Dobo et al. Spunbond fibers are quenched and generally not tacky on the surface when they enter the draw unit, or when they are deposited onto a collecting surface. Spunbond fibers are generally continuous and may have average diameters larger than 7 microns, often between about 10 and 30 microns.

The term “meltblown fibers” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than 10 microns in diameter, and are generally self bonding when deposited onto a collecting surface. Meltblown fibers used in the invention are desirably substantially continuous.

The term “polymer” generally includes but is not limited to, homopolymers, copolymers, including block, graft, random and alternating copolymers, terpolymers, etc., and blends and modifications thereof. Furthermore, unless otherwise specifically limited, the term “polymer” shall include all possible geometrical configurations of the material. These configurations include, but are not limited to isotactic, syndiotactic and atactic symmetries.

The term “substantially continuous filaments or fibers” refers to filaments or fibers prepared by extrusion from a spinnerette, including without limitation spunbonded and meltblown fibers, which are not cut from their original length prior to being formed into a nonwoven web or fabric. Substantially continuous filaments or fibers may have lengths ranging from greater than about 15 cm to more than one meter; and up to the length of the nonwoven web or fabric being formed.

The definition of “substantially continuous filaments or fibers” includes those which are not cut prior to being formed into a nonwoven web or fabric, but which are later cut when the nonwoven web or fabric is cut.

The term “staple filaments or fibers” means filaments or fibers which are natural or which are cut from a manufactured filament prior to forming into a web, and which have a length ranging from about 0.1-15 cm, more commonly about 0.2-7 cm.

The term “fiber” or “fibrous” is meant to refer to a particulate material wherein the length to diameter ratio of such particulate material is greater than about 10. Conversely, a “nonfiber” or “nonfibrous” material is meant to refer to a particulate material wherein the length to diameter ratio of such particulate material is about 10 or less.

The term “thermoplastic” is meant to describe a material that softens when exposed to heat and which substantially returns to its original condition when cooled to room temperature.

The term “recover” or “retract” relates to a contraction of a stretched material upon termination of a biasing force following stretching of the material by application of the biasing force.

The term “garment” includes personal care garments, medical garments, and the like. The term “disposable garment” includes garments which are typically disposed of after 1-5 uses.

The term “personal care garment” includes diapers, training pants, swim wear, absorbent underpants, adult incontinence products, feminine hygiene products, and the like.

The term “medical garment” includes medical (i.e., protective and/or surgical) gowns, caps, gloves, drapes, face masks, and the like.

The term “in the vicinity of garment openings” refers to a targeted elastic region of the garment within about two inches, suitably within about one inch, of a garment opening, such as a leg or waist opening. An elastic band or zone is said to be “in the vicinity of a garment opening” if any portion of the elastic band or zone is within two inches, desirably within one inch of the garment opening.

The term “aligned with a garment opening” refers to a targeted elastic region (i.e., a high tension zone or TEM) that is parallel, or within plus or minus 30 degrees of parallel, to a garment edge defining a garment opening.

“Integral” is used to refer to various portions of a single unitary element rather than separate structures bonded to or placed with or placed near one another.

“Inward” and “outward” refer to positions relative to the center of an article, and particularly transversely and/or longitudinally closer to or away from the longitudinal and transverse center of the article.

The terms “breathable layer” or “breathable film” refer to a film, laminate, or other material having a water vapor transmission rate (“WVTR”) of at least about 300 grams/m²-24 hours, using the WVTR Test Procedure described herein. Breathable materials typically rely on molecular diffusion of vapor, and are substantially liquid impermeable.

The term “film” refers to a thermoplastic film made using a film extrusion and/or foaming process, such as a cast film or blown film extrusion process. The term includes apertured films, slit films, and other porous films which constitute liquid transfer films, as well as films which do not transfer liquid. The term also includes film-like materials that exist as open-celled foams.

The term “series” refers to a set including one or more elements.

### DETAILED DESCRIPTION OF THE PRESENTLY PREFERRED EMBODIMENTS

The principles of this invention can be applied to a wide variety of garments, including disposable garments, having a targeted elastic zone in the vicinity of at least one garment opening. Examples include diapers, training pants, certain feminine hygiene products, adult incontinence products, other personal care or medical garments, and the like. For ease of explanation, the following description is in terms of a child training pant having a targeted elastic material, in this case a targeted elastic laminate, used for containment flaps and a waist dam.

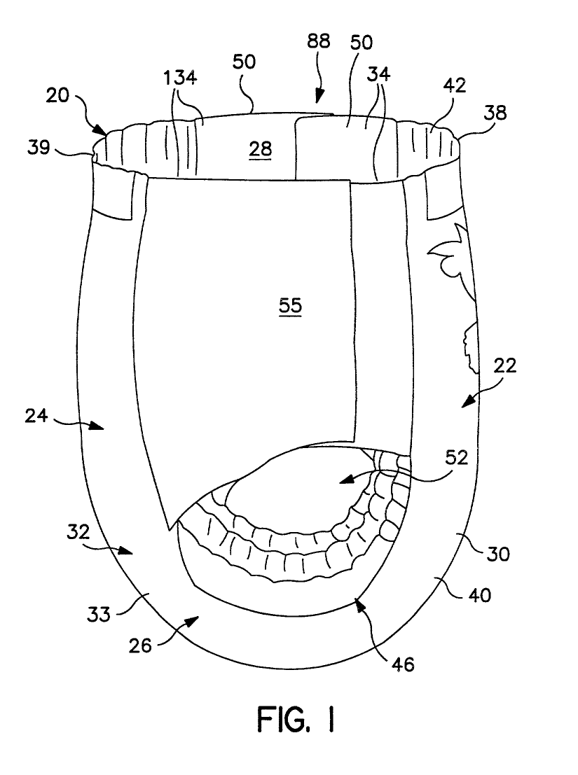

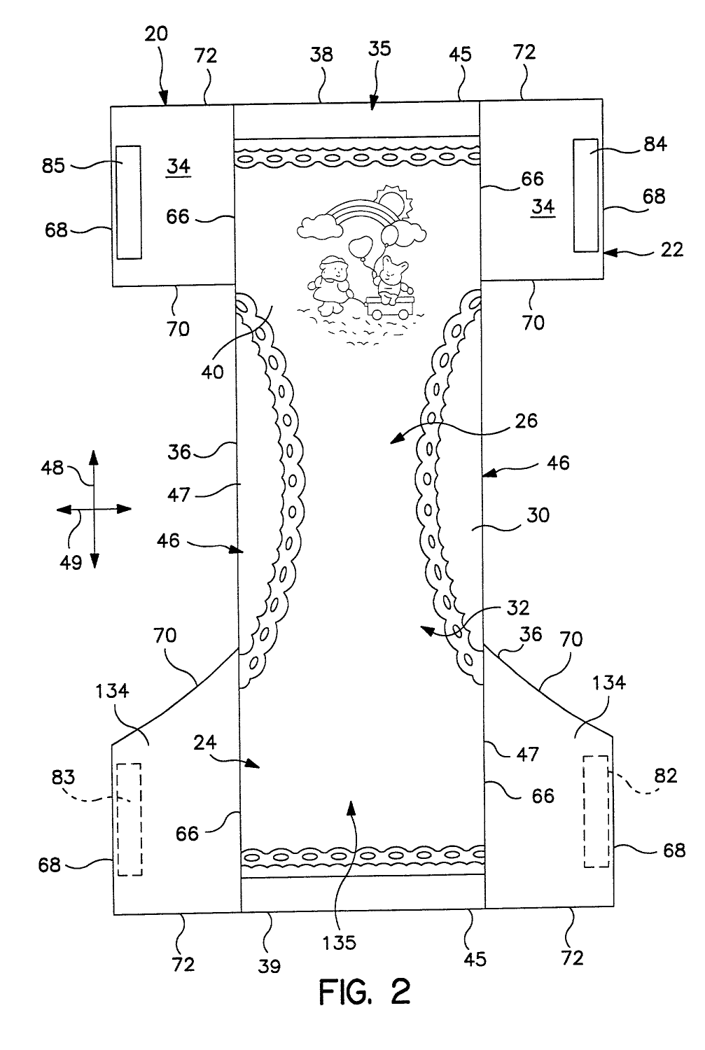

Referring to FIG. 1, a disposable absorbent garment 20, such as a child training pant, includes an absorbent chassis 32 and a fastening system 88. The absorbent chassis 32 defines a front waist region 22, a back waist region 24, a crotch region 26 interconnecting the front and back waist regions, an inner surface 28 which is configured to contact the wearer, and an outer surface 30 opposite the inner surface which is configured to contact the wearer's clothing. With additional reference to FIGS. 2 and 3, the absorbent chassis 32 also defines a pair of transversely opposed side edges 36 and a pair of longitudinally opposed waist edges, which are designated front waist edge 38 and back waist edge 39. The front waist region 22 is contiguous with the front waist edge 38, and the back waist region 24 is contiguous with the back waist edge 39. The chassis 32 defines waist opening 50 and two opposing leg openings 52.

The illustrated absorbent chassis 32 comprises a rectangular absorbent composite structure 33, a pair of transversely opposed front side panels 34, and a pair of transversely opposed back side panels 134. The composite structure 33 and side panels 34 and 134 may be integrally formed or comprise two or more separate elements, as shown in FIG. 1. The illustrated composite structure 33 comprises an outer cover 40, a bodyside liner 42 (FIGS. 1 and 3) which is connected to the outer cover in a superposed relation, an absorbent assembly 44 (FIG. 3) which is located between the outer cover and the bodyside liner, and a part of containment flaps 46 (FIG. 3). The rectangular composite structure 33 has opposite linear end edges 45 that form portions of the front and back waist edges 38 and 39, and opposite linear side edges 47 that form portions of the side edges 36 of the absorbent chassis 32 (FIGS. 2 and 3). For reference, arrows 48 and 49 depicting the orientation of the longitudinal axis and the transverse axis, respectively, of the training pant 20 are illustrated in FIGS. 2 and 3.

With the training pant 20 in the fastened position as illustrated in FIG. 1, the front and back waist regions 22 and 24 are joined together to define a three-dimensional pant configuration having a waist opening 50 and a pair of leg openings 52. The front waist region 22 includes the portion of the training pant 20 which, when worn, is positioned on the front of the wearer while the back waist region 24 comprises the portion of the training paint which, when worn, is positioned on the back of the wearer. The crotch region 26 of the training pant 20 includes the portion of the training pant which, when worn, is positioned between the legs of the wearer and covers the lower torso of the wearer. The front and back side panels 34 and 134 comprise the portions of the training pant 20 which, when worn, are positioned on the hips of the wearer.

The front waist region 22 of the absorbent chassis 32 includes the transversely opposed front side panels 34 and a front center panel 35 (FIGS. 2 and 3) positioned between and interconnecting the side panels. The back waist region 24 of the absorbent chassis 32 includes the transversely opposed back side panels 134 and a back center panel 135 (FIGS. 2 and 3) positioned between and interconnecting the side panels. The waist edges 38 and 39 of the absorbent chassis 32 are configured to encircle the waist of the wearer when worn and provide the waist opening 50 which defines a waist perimeter dimension. Portions of the transversely opposed side edges 36 in the crotch region 26 generally define the leg openings 52.

In the embodiment shown in FIG. 1, the front and back side panels 34 and 134 are fastened together by fastening system 88 to form collective side panels 55 (with each collective side panel 55 including a front side panel 34 and back side panel 134). In alternate embodiments, the collective side panels 55 may be single-piece side panels, or may include more than one piece permanently joined together. The transversely opposed front side panels 34 and transversely opposed back side panels 134 can be permanently bonded to the composite structure 33 of the absorbent chassis 32 in the respective front and back waist regions 22 and 24. More particularly, as shown best in FIGS. 2 and 3, the front side panels 34 can be permanently bonded to and extend transversely beyond the linear side edges 47 of the composite structure 33 in the front waist region 22 along attachment lines 66, and the back side panels 134 can be permanently bonded to and extend transversely beyond the linear side edges of the composite structure in the back waist region 24 along attachment lines 66. The side panels 34 and 134 may be attached using attachment means known to those skilled in the art such as adhesive, thermal or ultrasonic bonding. The side panels 34 and 134 can also be formed as a portion of a component of the composite structure 33, such as the outer cover or the bodyside liner. The fastening system 88 may include a plurality of fastener tabs 82, 83, 84 and 85, which can be known hook-and-loop fastener members, or other types of mechanical fasteners or adhesive fasteners. Alternatively, the front and back side panels 34, 134 can be permanently bonded together.

The illustrated side panels 34 and 134, in FIGS. 2 and 3, each define a distal edge 68 that is spaced from the attachment line 66, a leg end edge 70 disposed toward the longitudinal center of the training pant 20, and a waist end edge 72 disposed toward a longitudinal end of the training pant. The leg end edge 70 and waist end edge 72 extend from the side edges 47 of the composite structure 33 to the distal edges 68. The leg end edges 70 of the side panels 34 and 134 form part of the side edges 36 of the absorbent chassis 32. In the back waist region 24, the leg end edges 70 are desirably although not necessarily angled relative to the transverse axis 49 to provide greater coverage toward the back of the pant as compared to the front of the pant. The waist end edges 72 are desirably parallel to the transverse axis 49. The waist end edges 72 of the front side panels 34 form part of the front waist edge 38 of the absorbent chassis 32, and the waist end edges 72 of the back side panels 134 form part of the back waist edge 39 of the absorbent chassis.

In particular embodiments for improved fit and appearance, the side panels 34 and 134 desirably have an average length dimension measured parallel to the longitudinal axis 48 that is about 20 percent or greater, and particularly about 25 percent or greater, of the overall length dimension of the absorbent article, also measured parallel to the longitudinal axis 48. For example, in training pants having an overall length dimension of about 54 centimeters, the side panels 34 and 134 desirably have an average length dimension of about 10 centimeters or greater, such as about 15 centimeters. While each of the side panels 34 and 134 extend from the waist opening 50 to one of the leg openings 52, the back side panels 134 have a continually decreasing length dimension moving from the attachment line 66 to the distal edge 68, as is best shown in FIGS. 2 and 3.

The absorbent chassis 32 is configured to contain and/or absorb any body exudates discharged from the wearer. For example, the absorbent chassis 32 desirably includes the pair of containment flaps 46 which are configured to provide a barrier to the transverse flow of body exudates. The elasticized containment flaps 46 define an unattached, gasket-like edge 90 which assumes an upright, generally perpendicular configuration in at least the crotch region 26 of the training pant 20 to form a gasket-like seal against the wearer's body (FIG. 3). A closure edge 92, located on the flap 46 opposite the gasket-like edge 90, is also elasticized. Elastic tension along the gasket-like edge 90 is desirably higher than the elastic tension along the closure edge 92. Additionally, or in the alternative, the gasket-like edge 90 is a low stretch zone and the closure edge 92 is a high stretch zone. The containment flaps 46 can be located along the transversely opposed side edges of the absorbent chassis 32, and can extend longitudinally along the entire length of the absorbent chassis or may only extend partially along the length of the absorbent chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference. Suitably, the containment flaps 46 are lateral extensions of the absorbent chassis 32 such that the containment flaps 46 and at least one layer of the chassis 32 comprise a single piece of material.

Referring to FIGS. 1-3, in accordance with the invention, the containment flaps 46, suitably continuous with the chassis 32, each include a targeted elastic material (TEM) including an elasticized, low tension and/or high stretch zone 130 in the vicinity of (and aligned with) leg openings 52, and a narrow, band-like high tension and/or low stretch zone 131 in the vicinity of (and aligned with) the unattached, gasket-like edges 90 of the containment flaps 46 thereby creating a gasket at the gasket-like edges 90 of the containment flaps 46 (FIG. 3). The containment flaps 46 can be separate, attached pieces (as shown in FIGS. 1 and 2), or can be an extension of the outer cover 40, as shown in FIG. 3. The dotted lines in FIG. 3 indicate the boundaries between the low tension and/or high stretch zone 130 and the high tension and/or low stretch zone 131, which boundaries are not visible to an observer. The low tension and/or high stretch zone 130 and the high tension and/or low stretch zone 131 are desirably spaced apart, as shown in FIG. 3. From the standpoint of the observer, the TEM forming the containment flaps 46 appears as a homogeneous, integrated material.

The high tension and/or low stretch zone 131 exhibits greater elastic tension and/or elongation than the low tension and/or high stretch zone 130 of the containment flaps 46, without requiring the use of separately manufactured and attached elastic materials. Furthermore, desired spacing between the high tension and/or low stretch zone 131 and the low tension and/or high stretch zone 130 allows the zones 131 and 130 to stretch independently of one another so as not to constrain elongation capacity of either zone 131 and 130. For example, spacing between the low tension and/or high stretch zone 130 and the high tension and/or low stretch zone 131 should be at least 0.1 cm, suitably at least 1.0 cm, desirably at least 2.0 cm, alternatively at least 3.0 cm. Maximum spacing between the low tension and/or high stretch zone 130 and the high tension and/or low stretch zone 131 is typically no greater than about 10 cm, and may be smaller depending on the garment type and size.

To further enhance containment and/or absorption of body exudates, the training pant 20 desirably includes a waist dam having a front waist dam portion 54 and a rear waist dam portion 56 (FIG. 3). The waist dam portions 54 and 56 are suitably continuous with the chassis 32, and each include a targeted elastic material (TEM), much like the TEM used in the containment flaps 46. The waist dam portions 54 and 56 include an elasticized, low tension and/or high stretch zone 132 a slight distance below the waist edges 38 and 39, respectively, and a narrow, band-like high tension and/or low stretch zone 133 in the vicinity of (and aligned with) the waist edges 38 and 39, respectively, thereby creating a gasket at gasket-like edges 94 of the waist dam portions 54 and 56 (FIG. 3). The waist dam portions 54 and 56 can be separate, attached pieces, or can be extensions of the outer cover 40, as shown in FIG. 3. The dotted lines in FIG. 3 indicate the boundaries between the low tension and/or high stretch zone 132 and the high tension and/or low stretch zone 133, which boundaries are not visible to an observer. The low tension and/or high stretch zone 132 and the high tension and/or low stretch zone 133 are desirably spaced apart, as shown in FIG. 3. From the standpoint of the observer, the TEM forming the waist dam portions 54 and 56 appears as a homogeneous, integrated material.

As in the variable tension and/or stretch zones 130 and 131 of the containment flaps 46, the high tension and/or low stretch zone 133 exhibits greater elastic tension and/or less elongation than the low tension and/or high stretch zone 132 of the waist dam portions 54 and 56, without requiring the use of separately manufactured and attached elastic materials. Furthermore, desired spacing between the high tension and/or low stretch zone 133 and the low tension and/or high stretch zone 132 allows the zones 133 and 132 to stretch independently of one another so as not to constrain elongation capacity of either zone 133 and 132. For example, spacing between the low tension and/or high stretch zone 132 and the high tension and/or low stretch zone 133 should be at least 0.1 cm, suitably at least 1.0 cm, desirably at least 2.0 cm, alternatively at least 3.0 cm. Maximum spacing between the low tension and/or high stretch zone 132 and the high tension and/or low stretch zone 133 is typically no greater than about 10 cm, and may be smaller depending on the garment type and size.

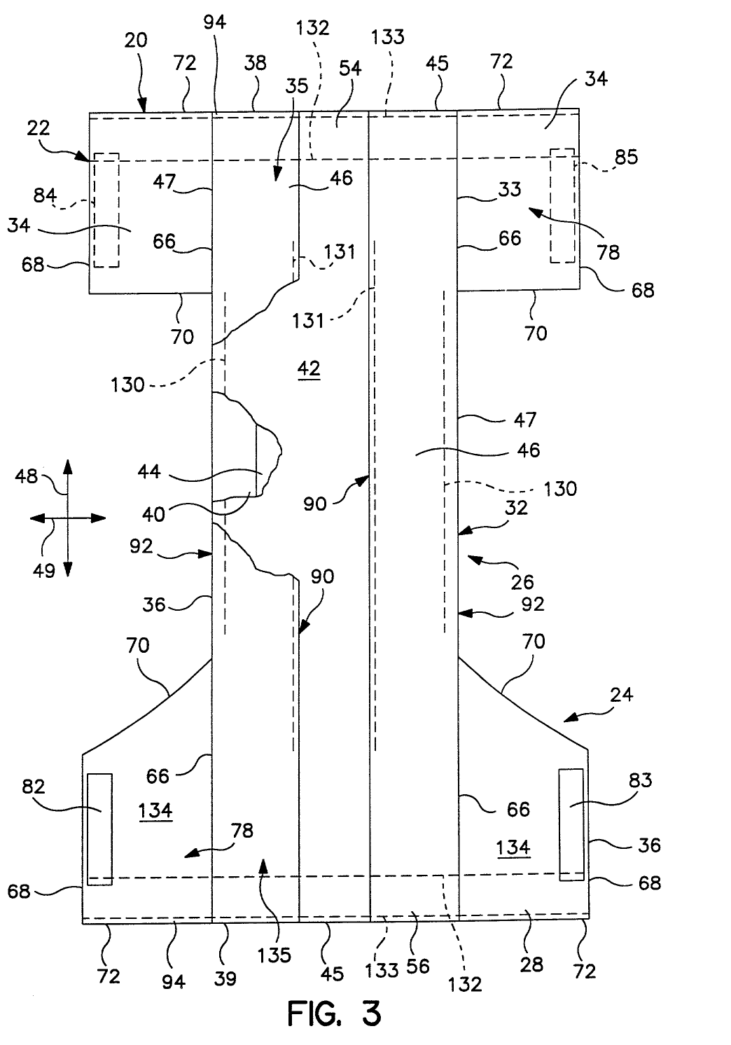

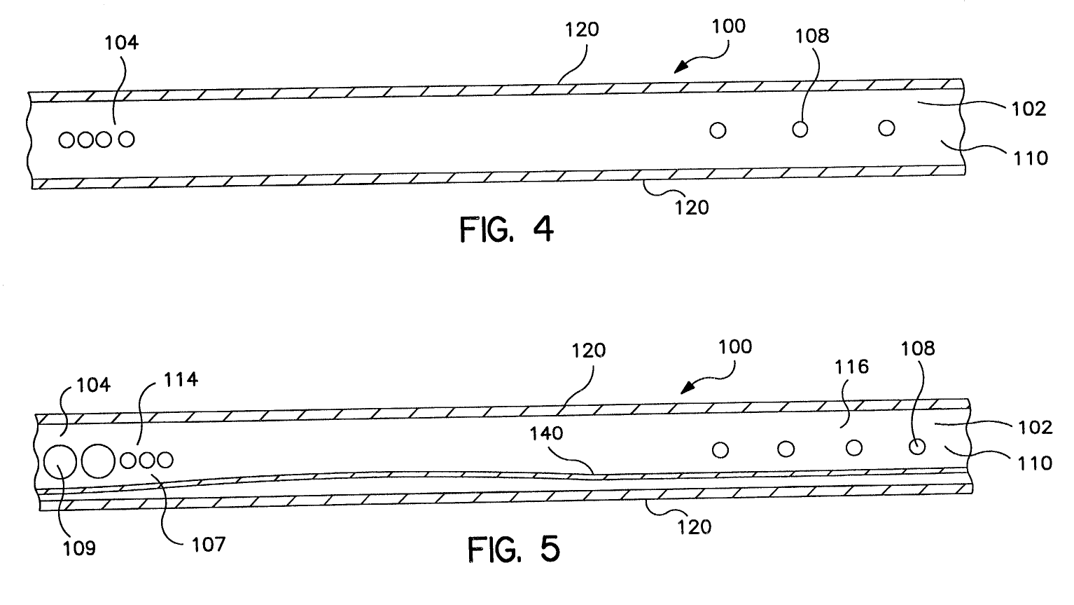

The containment flaps 46 and the waist dam portions 54 and 56 are manufactured from a targeted elastic material. Various embodiments of targeted elastic materials include the targeted elastic laminate materials shown in FIGS. 4-7. Referring to FIG. 4, TEL 100 (shown in sectional view, with the layers expanded apart from each other for clarity) includes a nonwoven layer 110 of elastomeric polymer filaments made from a single elastic polymer or polymer blend, laminated to at least one, suitably two outer facing layers 120. TEL 100 includes a low tension and/or high stretch end zone 102 (which may correspond to the elasticized low tension and/or high stretch zones 130 and 132 in the containment flaps 46 and the waist dam portions 54 and 56 of FIG. 3, respectively) and a high tension and/or low stretch end zone 104 (which may correspond to the high tension and/or low stretch zones 131 and 133 in FIG. 3). In the embodiment of FIG. 4, the polymer filaments 108 in the low tension and/or high stretch zone 102 are spaced further apart and, thus define a relatively low basis weight per unit area of nonwoven layer 110. The polymer filaments 108 in the high tension and/or low stretch zone 104 are spaced more closely together and, thus, define a higher basis weight per unit area of nonwoven layer 110. Except for the spacing between filaments (and the resulting variation in nonwoven web basis weight), the polymer filaments 108 may be identical in size and composition. The elastomeric nonwoven layer 110 may be stretched in the machine direction (i.e., a direction parallel to the longitudinal orientation of filaments 108) prior to bonding the nonwoven layer 110 to the facing layer 120 using processes as described below. After the layers are bonded together, the laminate may be relaxed (allowing retraction) and extended again as needed. As mentioned, spacing between the low tension and/or high stretch zone 102 and the high tension and/or low stretch zone 104 is desirable. Examples of such spacing are illustrated in FIGS. 4-7.

The TEL 100, when viewed by itself or in garment 20, would exhibit no visible perception of the high tension and/or low stretch zone 104 as distinguished from the low tension and/or high stretch zone 102. Instead, TEL 100 would appear as a homogeneous material, particularly when viewed from an outer surface of one of the facing layers 120. Yet the low tension and/or high stretch zone 102 and the high tension and/or low stretch zone 104 may function and perform as elastic leg bands and gaskets (i.e., may exhibit elasticity and elastic tension as would be provided by separately manufactured elastic bands). In order to accomplish this, the TEL 100 need only be sized and positioned in garment 20 so that the low tension and/or high stretch zone 102 and the high tension and/or low stretch zone 104 of the TEL are aligned with the leg openings of the garment. The TEL 100 may be used to manufacture containment flaps 46 and/or waist dam portions 54 and 56 as shown, or may be used in larger portions of the garment, in alternative embodiments.

FIGS. 5-7 illustrate alternative embodiments of TEL materials which can be used to make the garment of FIGS. 1-3. In FIG. 5, polymer filaments 108 in low tension and/or high stretch zone 102 have relatively small diameters, and relatively large spacings between them. Polymer filaments 109 in outer high tension and/or low stretch zone 104 have larger diameters than filaments 108, thus defining a higher nonwoven basis weight in zone 104. Polymer filaments 107 in inner high tension and/or low stretch zone 114 have similar diameters but less inter-filament spacing than polymer filaments 108, again defining a higher nonwoven basis weight in zone 114 than in low tension and/or high stretch zone 102. A breathable barrier layer 140 is inserted between one of the facing layers 120 and each of the polymer filaments 107, 108 and 109 extending through both the low and high tension and/or stretch zones 102 and 104.

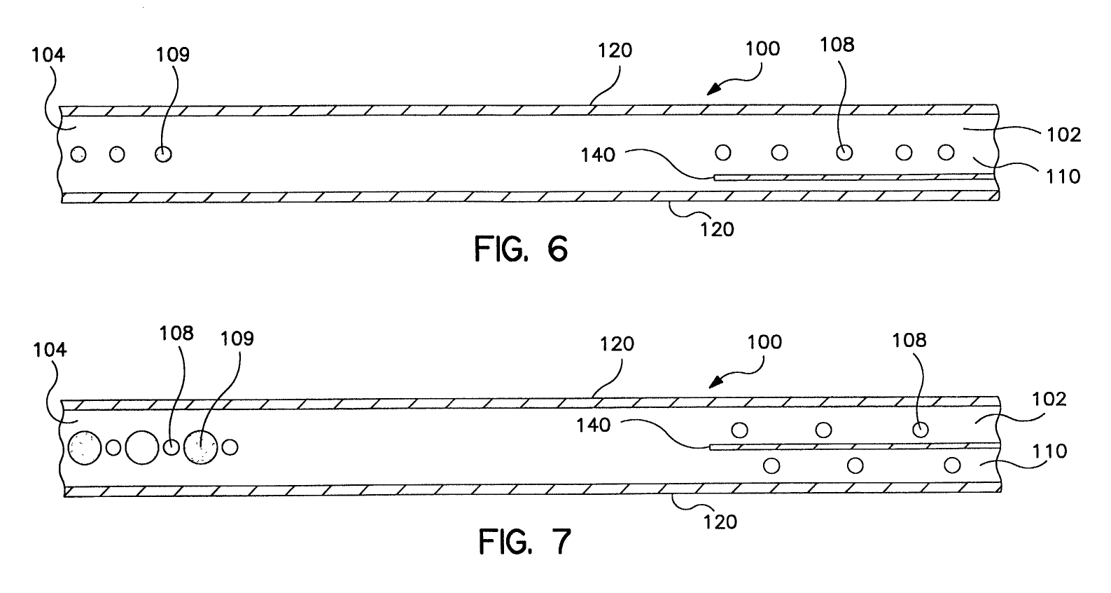

The barrier layer 140 can be made from a liquid impermeable polymer, suitably a stretchable olefin polymer, such as an olefinic co-polymer of polyethylene. Barrier film is desirably breathable to moisture vapor. Moisture vapor breathability can be achieved using known techniques, such as by mixing the polymer with 25-75% by weight of a particulate inorganic filler, forming a film from the mixture, and stretching the film by 1.5-7 times in at least the machine direction to cause voids and micropores to form around the filler particles. The barrier film 140 suitably has a water vapor transmission rate (WVTR) of at least 300 grams/m²-24 hours, desirably at least 1200 grams/m²-24 hours, alternatively at least 2000 grams/m²-24 hours, measured using the procedure described below. Barrier film may be present in both the low and high tension and/or stretch zones 102 and 104 as shown in FIG. 5, or in just the low tension and/or high stretch zone 102, as shown in FIGS. 6 and 7.

In the TEL of FIG. 6, the low and high tension and/or stretch zones 102 and 104 are accomplished by forming the nonwoven layer 110 with two different elastic polymers or polymer blends, each one having a different elastic tension when stretched. In addition, a breathable barrier layer 140 is inserted between the filaments 108 and one of the facing layers 120 in the low tension and/or high stretch zone 102. The breathable barrier layer 140 allows moisture vapor to pass through while blocking the flow of liquid through the layer 140.

In FIG. 6, the filaments 108 in low tension and/or high stretch zone 102 are formed from a first elastic polymer or polymer blend having lower elastic tension and/or higher elongation. The filaments 109 in high tension and/or low stretch zone 104 are formed from a second elastic polymer or polymer blend having higher elastic tension and/or lower elongation. Because different elastic polymers or polymer blends are used, the nonwoven layer 110 may have the same or different basis weights, the same or different filament sizes, and the same or different filament spacings in the low and high tension and/or stretch zones 102 and 104. The barrier film 140 in FIG. 6 is only present in the low tension and/or high stretch zone 102, between filaments 108 and lower facing layer 120.

The laminates of FIGS. 4-5 may each be produced by extruding the filaments 107 and 108 of nonwoven layer 110 from a single die, having die plate openings sized and spaced to correspond to the desired filament sizes and spacing, or from different dies. The laminate of FIG. 6 may be produced by extruding filaments from either the same die fed by two or more polymer extruders, or from different dies for each polymer. Some of the processes described below illustrate how this is accomplished. In the laminate of FIG. 7, the nonwoven layer 110 may be formed by extruding two narrower bands of higher tension and/or lower stretch filaments 109 over a single wider band of lower tension and/or higher stretch filaments 108, using different dies and extruders. The result, shown in FIG. 7, is that low tension and/or high stretch zone 102 contains only low tension and/or high stretch filaments formed of a first elastic polymer or polymer blend. High tension and/or low stretch zone 104 contains both high tension and/or low stretch filaments 109 formed of a second elastic polymer or polymer blend, and low tension and/or high stretch filaments 108. In addition, the breathable barrier layer 140 is inserted between two layers of the filaments 108 in the low tension and/or high stretch zone 102. As in the embodiment in FIG. 6, the breathable barrier layer 140 allows moisture vapor to pass through while blocking the flow of liquid through the layer 140.

In TEL 100, low tension and/or high stretch zone 102 may have a first elastic tension, measured at 50% elongation of the filaments, and high tension and/or low stretch zone 104 may have a second elastic tension higher than the first tension, measured at the same elongation. At 50% elongation of the TEL 100 (in the machine direction, parallel to filament orientation), high tension and/or low stretch zone 104 may have an elastic tension at least 10% greater, suitably at least 50% greater, desirably 100-800% greater, alternatively about 125-500% greater, or as another alternative about 150-300% greater than the low tension and/or high stretch zone 102. Elastic tension may be measured, for instance, using an MTS Sintec Model 1/s, sold by MTS in Research Triangle Park, N.C., with a crosshead speed set to 500 mm/min. Samples having a 3-inch width and 6-inch length can be used, with 3 inches of the length clamped inside the jaws (leaving 3 inches of length for testing). The tension of each high and low tension region can be measured after the portion of the TEL laminate being tested is held in the extended condition (in the machine direction of the TEL) for 60 seconds.

In the TEL embodiments where the low and high tension and/or stretch zones are formed from nonwoven web sections having different basis weights (FIGS. 4-5), the nonwoven basis weights in the high tension and/or low stretch zone 104 may be at least 10% greater, suitably at least 50% greater, desirably 100-800% greater, alternatively 125-500% greater, or as another alternative 200-400% greater than the nonwoven basis weight in the low tension and/or high stretch zone 102. For instance, the nonwoven in the low tension and/or high stretch zone 102 may have a basis weight of about 2-14 grams per square meter (gsm), desirably about 4-12 gsm. In the high tension and/or low stretch zone 104, the nonwoven basis weight may be about 10-32 gsm, desirably about 12-30 gsm. If the higher and lower basis weights are achieved using spinning holes of different frequency in the die, resulting in a higher areal density of filaments in the high tension and/or low stretch regions and lower areal density of filaments in the low tension and/or high stretch region, then the higher areal density may be at least 10% greater, suitably at least 50% greater, desirably 100-800% greater, alternatively 125-500% greater, or as another alternative 200-400% greater than the lower areal density. The filament density in each zone may range from about 4-40 filaments per square inch (fsi), suitably about 12-30 fsi, measured perpendicular to the length of the filaments.

If the higher and lower basis weights are achieved using filaments of higher and lower diameters, as in FIG. 5, the higher diameter filaments 109 may have diameters at least 5% higher, suitably at least 20% higher, desirably 40-300% higher, alternatively 50-125% higher, or as another alternative 75-100% higher than the lower diameter filaments 108. The filament diameters in each zone may range from about 0.010-0.040 inch, suitably about 0.020-0.032 inch.

If the higher and lower tension zones are formed using nonwoven filaments 108 and 109 of different elastic polymer composition, as shown in FIG. 6, then the different elastic polymers or polymer blends should be selected to give the desired higher elastic tension and/or lower elongation in the high tension and/or low stretch zone 104 and the desired lower elastic tension and/or greater elongation in the low tension and/or high stretch zone 102. The nonwoven basis weights in the different zones may be the same or different, and may be adjusted, along with the polymer compositions, to achieve the desired elastic tensions. When a polymer blend is used, the blend itself should exhibit the desired elastic tension and/or elongation, regardless of the properties of the individual components.

Materials suitable for use in preparing elastomeric filaments 108 and 109 in the low and high tension and/or stretch zones 102 and 104 include diblock, triblock, tetrablock or other multi-block elastomeric copolymers such as olefinic copolymers, including styrene-isoprene-styrene, styrene-butadiene-styrene, styrene-ethylene/butylene-styrene, or styrene-ethylene/propylene-styrene, which may be obtained from the Shell Chemical Company, under the trade designation KRATON® elastomeric resin; polyurethanes, including those available from E. I. Du Pont de Nemours Co., under the trade name LYCRA® polyurethane; polyamides, including polyether block amides available from Ato Chemical Company, under the trade name PEBAX® polyether block amide; polyesters, such as those available from E. I. Du Pont de Nemours Co., under the trade name HYTREL® polyester; and single-site or metallocene-catalyzed polyolefins having density less than about 0.89 grams/cc, available from Dow Chemical Co. under the trade name AFFINITY®.

A number of block copolymers can be used to prepare thermoplastic elastomeric filaments 108, 109 useful in this invention. Such block copolymers generally comprise an elastomeric midblock portion B and a thermoplastic endblock portion A. The block copolymers may also be thermoplastic in the sense that they can be melted, formed, and resolidified several times with little or no change in physical properties (assuming a minimum of oxidative degradation).

Endblock portion A may comprise a poly(vinylarene), such as polystyrene. Midblock portion B may comprise a substantially amorphous polyolefin such as polyisoprene, ethylene/propylene polymers, ethylene/butylene polymers, polybutadiene, and the like, or mixtures thereof.

Suitable block copolymers useful in this invention include at least two substantially polystyrene endblock portions and at least one substantially ethylene/butylene mid-block portion. A commercially available example of such a linear block copolymer is available from the Shell Chemical Company under the trade designation KRATON® G1657 elastomeric resin. Another suitable elastomer is KRATON® G2740.

Other suitable elastomeric polymers may also be used to make thermoplastic elastomeric filaments 108, 109. These include, without limitation, elastomeric (single-site or metallocene catalyzed) polypropylene, polyethylene and other alpha-olefin homopolymers and copolymers, having density less than about 0.89 grams/cc; ethylene vinyl acetate copolymers; and substantially amorphous copolymers and terpolymers of ethylene-propylene, butene-propylene, and ethylene-propylene-butene.

Single-site catalyzed elastomeric polymers (for example, constrained geometry or metallocene-catalyzed elastomeric polymers) are available from Exxon Chemical Company of Baytown, Texas, and from Dow Chemical Company of Midland, Mich. The single-site process for making polyolefins uses a single-site catalyst which is activated (i.e., ionized) by a co-catalyst.

Commercial production of single-site catalyzed polymers is somewhat limited but growing. Such polymers are available from Exxon Chemical Company of Baytown, Tex. under the trade name EXXPOL® for polypropylene based polymers and EXACT® for polyethylene based polymers. Dow Chemical Company of Midland, Mich. has polymers commercially available under the name ENGAGE®. These materials are believed to be produced using non-stereo selective single-site catalysts. Exxon generally refers to their single-site catalyst technology as metallocene catalysts, while Dow refers to theirs as “constrained geometry” catalysts under the name INSITE® to distinguish them from traditional Ziegler-Natta catalysts which have multiple reaction sites. Other manufacturers such as Fina Oil, BASF, Amoco, Hoechst and Mobil are active in this area and it is believed that the availability of polymers produced according to this technology will grow substantially in the next decade.

Elastic filaments 108 and 109 may also contain blends of elastic and inelastic polymers, or of two or more elastic polymers, provided that the blend exhibits elastic properties. The filaments may be substantially continuous or staple in length, but are desirably substantially continuous. Substantially continuous filaments have better elastic recovery than staple length filaments. Elastic filaments 107, 108 and 109 may be circular but may also have other cross-sectional geometries such as elliptical, rectangular, triangular or multi-lobal. In one embodiment, one or more of the filaments may be in the form of elongated, rectangular film strips produced from a film extrusion die having a plurality of slotted openings.

The barrier film 140 can be produced from any of the elastic polymer materials described above, or from other stretchable polymers. As explained above, barrier film 140 may contain about 25-75% by weight, suitably 40-70% by weight, of a particulate inorganic filler having a mean particle size of less than 10 microns, suitably 0.5-3 microns. Desired fillers include calcium carbonate, titanium dioxide, talc, and the like. When the polymer/filler combination is formed into a film and stretched, as explained above, the film acquires voids separated by thin polymer membranes. The membranes block liquid transfer but facilitate water vapor transfer.

The facing layer or layers 120 may each include a nonwoven web, for example a spunbonded web or a meltblown web, a woven web, or a film. Facing materials may be formed using conventional processes, including the spunbond and meltblowing processes described in the “DEFINITIONS.” For example, facing materials 120 may include a spunbonded web having a basis weight of about 0.1-4.0 osy, suitably 0.2-2.0 osy, desirably about 0.4-0.6 osy. The facing materials 120 may include the same or similar materials or different materials.

Suitably, the facing materials 120 are bonded to a nonwoven layer 110 (including the low and high tension zones thereof) using an adhesive, for example an elastomeric adhesive such as Findley H2525A, H2525 or H2096. Other bonding means well known to those having ordinary skill in the art may also be used to bond the facing materials 120 to filaments 108 and 109 of nonwoven layer 110, including thermal bonding, ultrasonic bonding, mechanical stitching and the like.

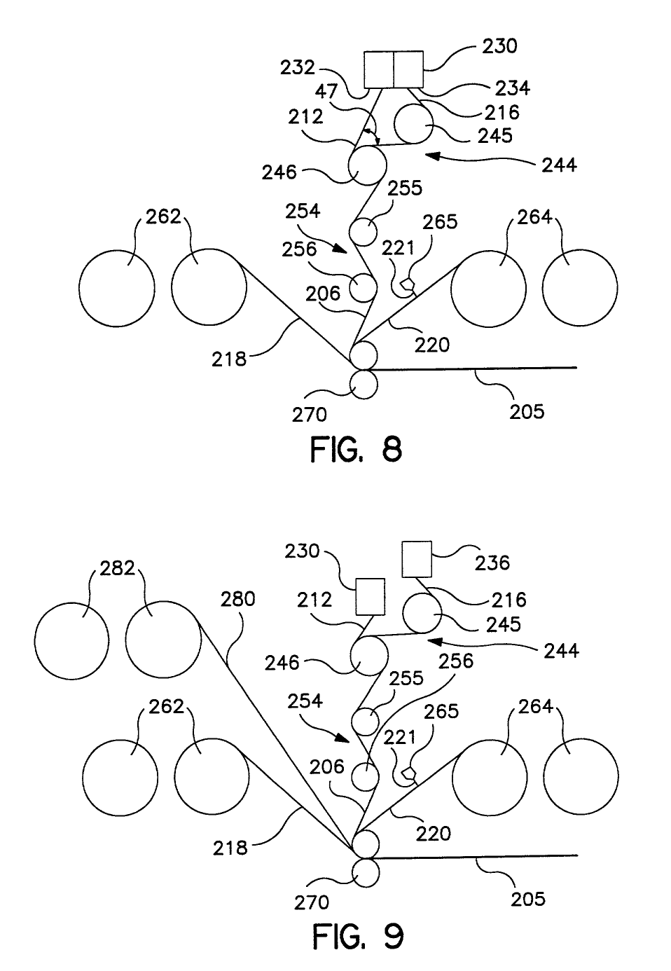

FIGS. 8-11 and 15 illustrate representative processes for making TEL materials. FIGS. 8 and 9 each illustrate a continuous vertical filament stretch-bond laminate (VF SBL) method. Referring to FIG. 8, an extruder (not shown) supplies molten elastomeric material to a first die 230. First die 230 includes different regions of spinning holes tailored to provide the nonwoven fabric 206 with higher and lower zones of elastic tension and/or stretch, having higher and lower basis weights or different polymer compositions as explained with respect to FIGS. 4-7.

Referring to FIG. 8, molten elastomeric material is extruded from first spin plate region 232 through spinning holes as a plurality of elastomeric first filaments 212. Similarly, a plurality of elastomeric second filaments 216 are extruded from second spin plate region 234 through spinning holes of different average diameter, different frequency, and/or different polymer composition. The resulting nonwoven web 206 has a higher elastic tension and/or lower stretch in the zone defined by second filaments 216, than in the zone defined by first filaments 212. After extruding, first and second filaments 212 and 216 are quenched and solidified.

In one embodiment, first and second filaments 212 and 216 are quenched and solidified by passing them over a first series of chill rolls 244. For instance, first filaments 212 may be contacted with chill roll 246. Second filaments 216, having a higher aggregate basis weight, may be passed over two chill rolls 245 and 246. Any number of chill rolls can be used. Suitably, chill rolls 245 and 246 have a temperature of about 40° F. to about 80° F.

After first and second filaments 212 and 216 are quenched and solidified, they are stretched or elongated. In one desired embodiment, first and second filaments 212 and 216 are stretched using a first series of stretch rolls 254. First series of stretch rolls 254 may include one or more individual stretch rolls 255, desirably at least two stretch rolls 255 and 256, as shown in FIG. 8. Stretch rolls 255 and 256 rotate at a speed greater than a speed at which chill rolls 245 and 246 rotate, thereby stretching the nonwoven fabric 206, including the zones of first and second filaments 212 and 216.

In one embodiment, each successive roll rotates at a speed greater than the speed of the previous roll. For example, referring to FIG. 8, chill roll 245 rotates at a speed “x”; chill roll 246 rotates at a speed greater than “x”, for example about “1.1x”; stretch roll 255 rotates at a still greater speed, for example about “1.15x”; second stretch roll 256 rotates at a still greater speed, for example about “1.25x” to about “2x”; and a third stretch roll 257 rotates at a still greater speed, for example about “2x” to about “7x.” As a result, first and second filaments 212 and 216 can be stretched by about 100% to about 800% of an initial length, suitably by about 200% to about 700% of an initial length.

After first and second filaments 212 and 216 are stretched, elastic nonwoven web 206 is laminated to a first facing material 218 and (alternatively) a second facing material 220. First facing material 218 is unwound from one of the rollers 262 and laminated to a first side of nonwoven web 206. Second facing material 220 is unwound from one of the rollers 264 and laminated to a second side of nonwoven web 206. As shown in FIG. 8, before second facing material 220 is laminated to a second side of elastic nonwoven web 206, at least a portion of second facing material 220 can be coated or sprayed with an elastomeric adhesive 221, such as Findley H2525A, H2525 or H2096, via an adhesive sprayer 265. The laminate material is then passed through nip rolls 270 (desirably smooth or patterned calender rolls) and is relaxed and/or retracted to produce a TEL 205. Other means for bonding the laminate material known to those having ordinary skill in the art may be used in place of nip roll 270.

FIG. 9 illustrates a VF SBL process similar to that of FIG. 8. In FIG. 9, instead of using a single spinnerette 230 having adjacent die regions for the high and low tension and/or stretch filament zones, two spinnerettes 230 and 236 are employed. First spinnerette 230 extrudes the first filaments 212. Second spinnerette 236 extrudes the second filaments 216. Again, the first and second spinnerettes differ as to the aggregate basis weights and/or polymer compositions of the elastomeric filaments produced. The second spinnerette 236 may have die openings of a) higher frequency and/or b) higher diameter, than the die openings of the first spinnerette 230. Additionally, an optional breathable barrier layer 280 is unwound from one of the rollers 282 and bonded between the first facing material 218 and at least the second filaments 216. Except for the use of two spinnerettes instead of one “hybrid” spinnerette, and the optional breathable barrier layer 280, the processes of FIGS. 8 and 9 are similar. In either case, the first filaments 212 and second filaments 216 ultimately converge to form a single elastic nonwoven web 206 having zones of higher and lower elastic tensions and/or stretch. The filaments 212 and 216 may converge in a spaced-apart fashion as shown in FIGS. 4-6, for instance, to produce zones of higher and lower tension and/or stretch. Alternatively, the bands of filaments 212 and 216 may have different widths such that a narrower layer or band of second filaments 216 is superimposed directly over a wider layer band of filaments 212, so that the higher tension and/or lower stretch zone occurs where the two layers coexist as exemplified in FIG. 7. In either process, the first filaments 212 and second filaments 216 may converge as shown, at the chill roll 246.

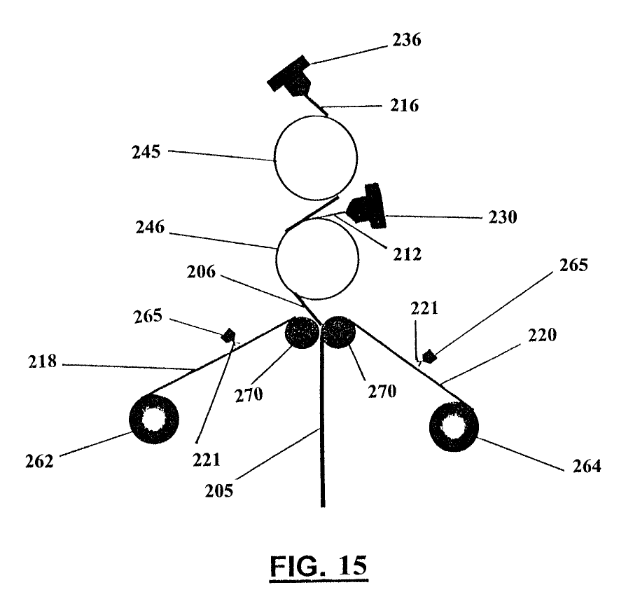

FIG. 15 illustrates a VF SBL process in which no stretch rolls 254 are used. Instead, first filaments 212 are extruded onto chill roll 246. Second filaments 216 are extruded onto chill roll 245, where the first filaments 212 and second filaments 216 converge to form a single elastic nonwoven layer 206 having zones of higher and lower elastic tensions. The first and second filaments 212, 216 are stretched between the chill rolls 245, 246 and the nip rolls 270. Except for the lack of stretch rolls 254, the processes of FIGS. 8 and 17 are similar. In either case, the elastic nonwoven layer 206 is laminated between a first facing layer 218 and a second facing layer 220 at the nip rolls 270. The resulting laminate is then relaxed and/or retracted to form TEL 205.

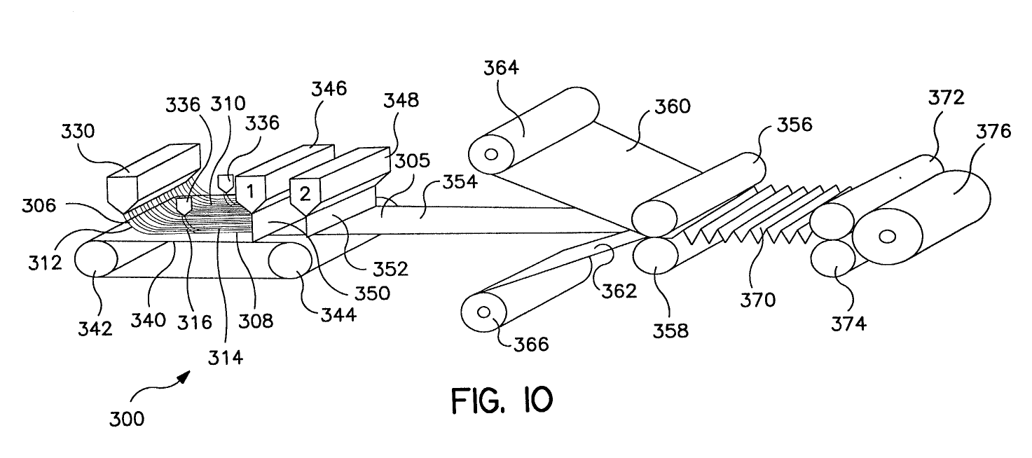

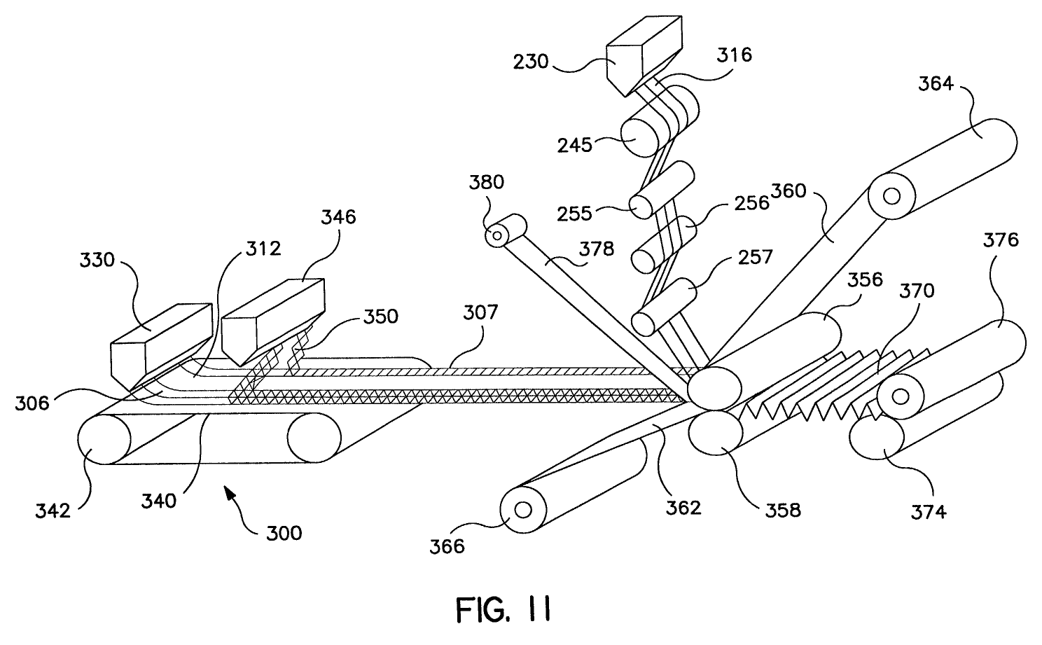

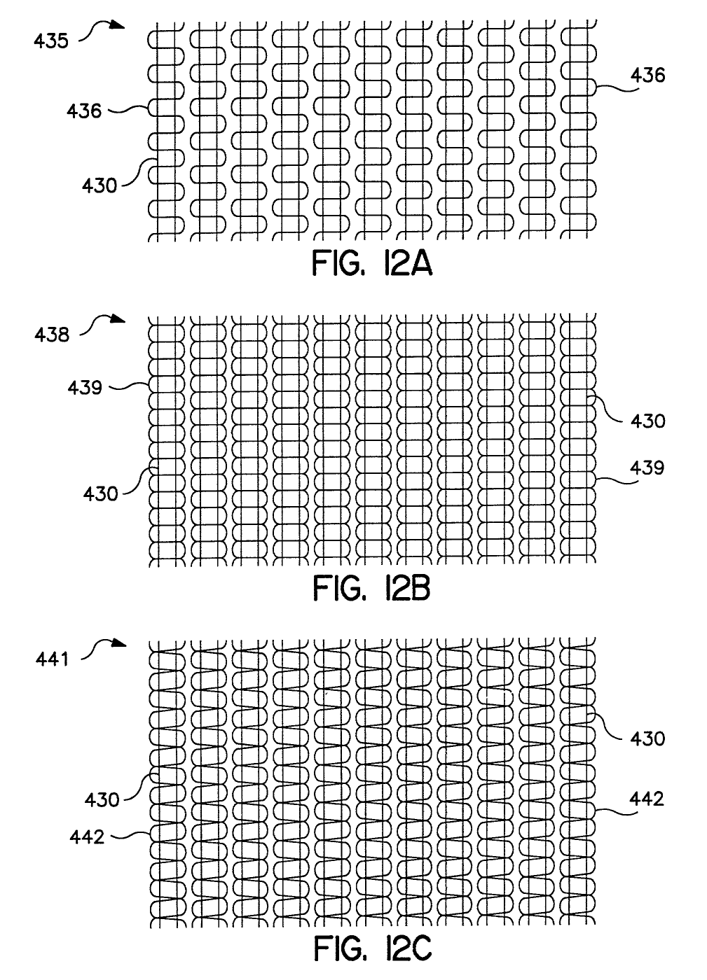

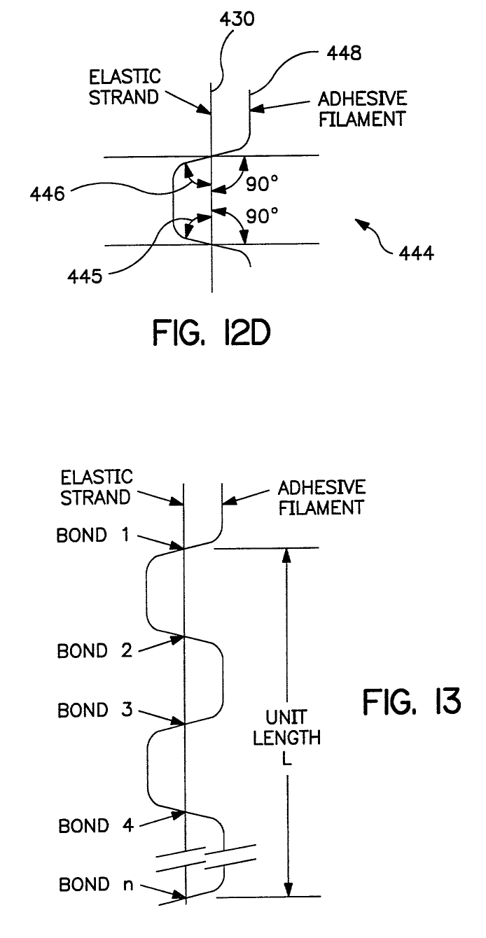

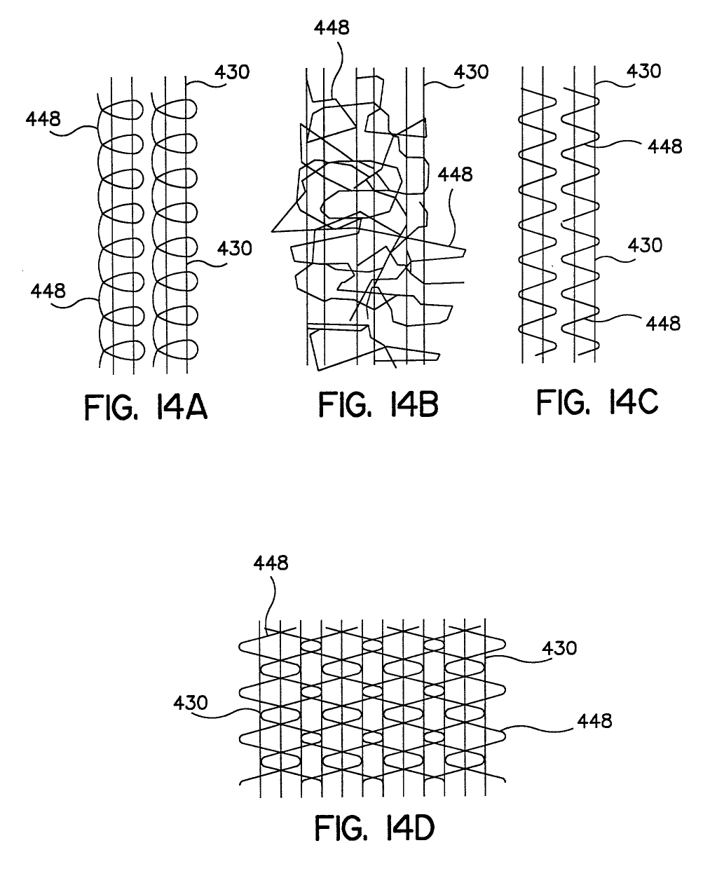

FIG. 10 illustrates a continuous horizontal filament stretch-bond laminate (CF SBL) process 300 for making TEL materials. A first extrusion apparatus 330 (which can be a spinnerette, as described above) is fed with an elastomeric polymer or polymer blend using one or more extruders (not shown). In various embodiments, the extrusion apparatus 330 can be configured to form a nonwoven layer 306 having zones of higher and lower elastic tension and/or stretch, as illustrated in FIGS. 4-7. In another embodiment, the extrusion apparatus 330 can be configured with die holes of uniform size and spacing, to yield a nonwoven layer 306 which has uniform elastic tension and/or stretch across its width. The nonwoven layer 306 contains filaments 312 which are substantially continuous in length. In this regard, the extrusion apparatus 330 may be a spinnerette. Suitably, apparatus 330 is a meltblowing spinnerette operating without the heated gas (e.g., air) stream which flows past the die tip in a conventional meltblowing process. Apparatus 330 extrudes filaments 312 directly onto a conveyor system, which can be a forming wire system 340 (i.e., a foraminous belt) moving clockwise about rollers 342. Filaments 312 may be cooled using vacuum suction applied through the forming wire system, and/or cooling fans (not shown). The vacuum may also assist in holding nonwoven layer 306 against the forming wire system.

In a desired embodiment, at least one, possibly one or more, second extrusion apparatus 336 are positioned downstream of the first extrusion apparatus 330. The second extrusion apparatus create one or more higher tension and/or low stretch zones in the nonwoven layer 306 by extruding filaments 316 of elastic material directly onto the nonwoven layer 306 in bands or zones which are narrower than the width of nonwoven layer 306. The second filaments 316 may be of the same elastic polymer construction as the first filaments 312. The extrusion of second filaments 316 over the first filaments 312 only in selected regions of layer 306, operates to create higher elastic tension and/or lower stretch zones 314 where the first and second filaments 312 and 316 coexist, and lower elastic tension and/or higher stretch zones 310 where the first filaments 312 exist alone. The first and second filaments 312 and 316 converge, and are combined in the forming conveyor 340 as it travels forward, to yield nonwoven layer 308 having at least one first zone 310 of lower elastic tension and/or higher stretch, and a second, outer zone 314 of higher elastic tension and/or lower stretch.

As explained above, nonwoven layer 308 can be produced either a) directly from spinnerette 330, which is configured to yield zones of higher and lower elastic tension and/or stretch similar to FIGS. 3-6, or b) through the combined effect of spinnerette 330 as a uniform or nonuniform die, and secondary spinnerettes 336 which increase the elastic tension and/or decrease the elongation in localized regions of layer 308 by extruding secondary filaments 316 onto layer 306, similar to the web in FIG. 7. In either case, the nonwoven layer 308 (including filaments 312 and 316) may be incidentally stretched and, to an extent, maintained in alignment by moving the foraminous conveyor 340 in a clockwise machine direction at a velocity which is slightly greater than the exit velocity of the filaments leaving the die.

To make the TEL 305, the elastic nonwoven layer 308 having higher and lower elastic tension and/or stretch zones is reinforced with one or more elastomeric meltblown layers made of the same or different elastic polymer material. Referring to FIG. 10, meltblowing extruders 346 and 348 are used to form meltblown layers 350 and 352 onto one side of layer 308, resulting in TEL 305. The meltblown layer or layers may act as structural facing layers in the laminate, and/or may act as tie layers if it is desired to add still more layers to the laminate.

Then, if it is desired to convert the TEL 305 into a stretch-bonded laminate, the TEL 305 may be stretched in a stretching stage 354 by pulling it between two nip rolls 356 and 358 which turn at a higher surface speed than the conveyor 340. At the same time, the facing layers 360 and 362 can be unwound from supply rollers 364 and 366, and laminated to the TEL 305 using the stretch roll assembly. To accomplish this dual purpose, the nip rolls 356 and 358 may be smooth or patterned calender rolls which use pressure to bond the materials 360, 305 and 362 together as well as stretch the TEL 305. Alternatively, both heat and pressure may be applied to bond the materials 360, 305 and 362 together. The resulting stretch-bonded laminate 370 may then be relaxed and/or retracted using nip rollers 372 and 374 that rotate at lower surface speed than calender rolls 358, and may be wound onto storage roll 376. The facing layers 360 and 362 may be any of the facing materials described above, and are suitably polyolefin-based spunbond webs.

FIG. 11 illustrates a hybrid 300 of a CF SBL process and a VF SBL process for making a stretch-bonded TEL 370. A first extrusion apparatus 330 is fed with an elastic polymer or polymer blend from one or more sources (not shown). Extrusion apparatus 330 may be any of the various devices described with respect to FIG. 10. Desirably, apparatus 330 is a meltblowing spinnerette operating without the heated gas (e.g., air) stream which flows past the die tip in conventional meltblowing processes. Apparatus 330 extrudes lower tension and/or higher stretch filaments 312 directly onto a conveyor system, which can be a forming wire system 340 (i.e., a foraminous belt) moving clockwise about rollers 342. Filaments 312 may be cooled using vacuum suction applied through the forming wire system, and/or cooling fans (not shown). The vacuum may also help hold the filaments against the forming wire system.

A meltblowing extruder 346 is used to add a reinforcing elastic meltblown layer 350 to the elastic filaments 312. Suitably, the meltblown layer 350 is made of the same elastic polymer as the low tension and/or high stretch filaments 312. The resulting laminate 307 travels forward on the conveyor.

Additionally, an optional breathable barrier layer 378 is unwound from supply roll 380 and joined between the laminate 307 and higher tension (i.e., higher basis weight) and/or lower stretch elastic filaments 316 in a higher tension and/or lower elongation region.

To make the higher tension and/or lower elongation region, a vertical filament die 230 extrudes the higher tension and/or low stretch elastic filaments 316 in a band which is narrower than the laminate 307 containing filaments 312. Filaments 316 pass around a chill roll 245, or a series of chill rolls, and a series of stretch rolls, for example three stretch rolls 255, 256 and 257, before being joined with laminate 307 between nip rolls 356 and 358, which are desirably smooth or patterned calender rolls. Simultaneously, facing layers 360 and 362 are unwound from supply rolls 364 and 366 and joined with the laminate between nip rolls 356 and 358 to make TEL 370. As TEL 370 is relaxed, it may assume the puckered configuration shown, due to retraction of high tension filaments 316 present in part of the laminate. TEL 370 may be flattened out between rolls 374 and 376, and wound onto roll 376.

The invention encompasses various types of garments in which a high tension and/or low stretch gasketing elastic zone is present in the vicinity of any one or more garment openings. Depending on the garment, high tension and/or low stretch gasketing zones of a TEM may encircle an entire garment opening or just a portion of the garment opening. In addition to the training pant 20, other types of garments on which this invention can be used include personal care garments, such as diapers, absorbent underpants, adult incontinence products, certain feminine hygiene articles, and swim wear. The high tension and/or low stretch gasketing elastic zones may be used in similar fashion in medical garments including, for instance, medical gowns, caps, gloves, drapes, face masks, and the like, where it is desired to provide a gasket in the vicinity of one or more garment openings without requiring a separately manufactured and attached elastic band. Furthermore, the high tension and/or low stretch gasketing elastic zone can be used around neck openings, arm openings, wrist openings, waist openings, leg openings, ankle openings, and any other opening surrounding a body part wherein fluid transfer resistance is desirable.

### WVTR TEST PROCEDURE

The following procedure is described for testing of the water vapor transmission rate (WVTR) for the breathable barrier films used in the invention. The WVTR is measured in a manner similar to ASTM Standard Test Method for Water Vapor Transmission of Materials, Designation E-96-80 as follows. For the purposes of the present invention, 3 inch diameter (76 mm) circular samples are cut from the test material and from a control material, CELGARD® 2500 (Hoechst Celanese Corporation). CELGARD 2500 is a 0.0025 cm thick film composed of microporous polypropylene. Two or three samples are prepared for each material. Test cups used for testing are cast aluminum, flanged, 5.1 centimeters deep and come with a mechanical seal and neoprene gasket. The cups are distributed by Thwing-Albert Instrument Company, Philadelphia, Pa., under the designation Vapometer cup no. 68-1. One hundred millimeters of distilled water is poured into each Vapometer cup, and each of the individual samples of the test materials and control material are placed across the top area of an individual cup. Screw-on flanges are tightened to form a seal along the edges of the cups leaving the associated test material or control material exposed to the ambient atmosphere over a 62 millimeter diameter circular area (an open, exposed area of about 30 cm²). The cups are then weighed, placed on a tray, and set in a forced air oven set at 100° F. (38° C.). The oven is a constant temperature oven with external air through it to prevent water vapor accumulation inside. A suitable forced air oven is, for example, a Blue M Power-O-Matic 60 oven distributed by Blue M Electric Co. of Blue Island, Ill. After 24 hours, the cups are removed from the oven and weighed. The preliminary, test WVTR value is calculated as follows:

Test WVTR=[(grams weight loss over 24 hours)×7571]÷24

The relative humidity within the oven is not specifically controlled. Under predetermined set conditions of 38° C. and ambient relative humidity, the WVTR for CELGARD 2500 has been determined to be 5000 g/m²-24 hours. Accordingly, CELGARD 2500 is run as a control sample with each test and the resulting values are corrected in accord with the variation of the control relative to its known WVTR.

While the embodiments of the invention described herein are presently preferred, various modifications and improvements can be made without departing from the spirit and scope of the invention. The scope of the invention is indicated in the appended claims, and all changes that fall within the meaning and range of equivalents are intended to be embraced therein.

What is claimed:

1. A garment having at least one opening for a body part, the garment comprising:
at least one material defining the opening and extending away from the opening; and
a fluid sealing gasket integral with the material and in the vicinity of the opening;
the material including at least one high tension zone defining the fluid sealing gasket, and at least one low tension zone away from the gasket;
wherein the fluid sealing gasket interfaces with the body part during use to resist fluid transfer across the gasket.

2. The garment of claim 1 wherein the material comprises a targeted elastic laminate including the low and high tension zones;
the low tension zone including a plurality of elastomeric first filaments;
the high tension zone including a plurality of elastomeric second filaments;
the laminate further including a first facing layer bonded to at least a first side of the low tension zone and a first side of the high tension zone.

3. The garment of claim 1 wherein the material further comprises a targeted elastic laminate including low and high stretch zones.

4. The garment of claim 2, wherein the elastomeric first and second filaments have different average filament sizes.

5. The garment of claim 2, wherein the elastomeric first and second filaments have different filament densities.

6. The garment of claim 2, further comprising a second facing layer bonded to a second side of the low tension zone and a second side of the high tension zone.

7. The garment of claim 2, further comprising a barrier layer between the first facing layer and the elastomeric first filaments.

8. The garment of claim 2, further comprising a barrier layer between at least two of the elastomeric first filaments.

9. The garment of claim 1, wherein the opening comprises a neck opening.

10. The garment of claim 1, wherein the opening comprises a wrist opening.

11. The garment of claim 1, wherein the opening comprises an arm opening.

12. The garment of claim 1, wherein the opening comprises a waist opening.

13. The garment of claim 1, wherein the opening comprises a leg opening.

14. The garment of claim 1, wherein the opening comprises an ankle opening.

15. The garment of claim 1, comprising a diaper.

16. The garment of claim 1, comprising a training pant.

17. The garment of claim 1, comprising a feminine hygiene article.

18. The garment of claim 1, comprising swim wear.

19. The garment of claim 1, comprising an absorbent underpant.

20. The garment of claim 1, comprising a medical gown.

21. The garment of claim 1, comprising a medical cap.

22. The garment of claim 1, comprising a medical glove.

23. The garment of claim 1, comprising a medical drape.

24. The garment of claim 1, comprising a medical face mask.

25. A garment having at least one opening for a body part, the garment comprising:
at least one material defining the opening and extending away from the opening; and
a fluid sealing gasket integral with the material and in the vicinity of the opening;
the material including at least one low stretch zone defining the fluid sealing gasket, and at least one high stretch zone away from the gasket;
wherein the fluid sealing gasket interfaces with the body part during use to resist fluid transfer across the gasket.

26. The garment of claim 25 wherein the material comprises a targeted elastic laminate including the low and high stretch zones;
the high stretch zone including a plurality of elastomeric first filaments;
the low stretch zone including a plurality of elastomeric second filaments;
the laminate further including a first facing layer bonded to at least a first side of the low stretch zone and a first side of the high stretch zone.

27. The garment of claim 26, further comprising a second facing layer bonded to a second side of the low stretch zone and a second side of the high stretch zone.

28. The garment of claim 26, further comprising a barrier layer between the first facing layer and the elastomeric first filaments.

29. A pant-like garment comprising:
a chassis defining a waist opening and two leg openings;
a fluid sealing gasket on the chassis and in the vicinity of at least one of the openings; and
a targeted elastic material in at least a portion of the chassis, the targeted elastic material including at least one high tension zone defining the fluid sealing gasket, and at least one low tension zone spaced apart from and inward from the high tension zone.

30. The pant-like garment of claim 29, further comprising a leg flap at least partially surrounding a leg opening, the leg flap including the targeted elastic material.

31. The pant-like garment of claim 30, wherein the leg flap comprises targeted elastic material attached to a portion of the chassis.

32. The pant-like garment of claim 30, wherein the leg flap comprises targeted elastic material integral with at least a portion of the chassis.

33. The pant-like garment of claim 29, further comprising a waist dam at least partially surrounding the waist opening, the waist dam including the targeted elastic material.

34. The pant-like garment of claim 33, wherein the waist dam comprises targeted elastic material attached to a portion of the chassis.

35. The pant-like garment of claim 33, wherein the waist dam comprises targeted elastic material integral with at least a portion of the chassis.

36. The pant-like garment of claim 29 wherein the targeted elastic material comprises a targeted elastic laminate including the low and high tension zones;
the low tension zone including a plurality of elastomeric first filaments;
the high tension zone including a plurality of elastomeric second filaments;
the laminate further including a facing layer bonded to a first side of the low tension zone and a first side of the high tension zone, and a second facing layer bonded to a second side of the low tension zone and a second side of the high tension zone.

37. The pant-like garment of claim 36, wherein the low tension zone further comprises a barrier layer.

38. The laminate of claim 37, wherein the barrier layer is present in both the low and high tension zones.

39. The pant-like garment of claim 37, wherein the barrier layer is bonded between elastomeric first filaments.

40. The pant-like garment of claim 37, wherein the barrier layer is bonded between the second facing layer and the second side of the low tension zone.

41. The pant-like garment of claim 36, wherein the low tension zone has a first basis weight and the high tension zone has a second basis weight higher than the first basis weight.

42. The pant-like garment of claim 36, wherein the elastomeric first filaments and the elastomeric second filaments comprise different polymer compositions.

43. The pant-like garment of claim 36, wherein the first filaments and the second filaments each comprise a base polymer selected from the group consisting of styrene-isoprene-styrene block copolymers, styrene-butadiene-styrene block copolymers, styrene-ethylene/butylene-styrene block copolymers, styrene-ethylene-propylene-styrene block copolymers, polyurethanes, elastomeric polyamides, elastomeric polyesters, elastomeric polyolefin homopolymers and copolymers, atactic polypropylenes, ethylene vinyl acetate copolymers, single-site or metallocene catalyzed polyolefins having a density less than about 0.89 grams/cc, and combinations thereof.

44. A pant-like garment comprising:
a chassis defining a waist opening and two leg openings;
a fluid sealing gasket on the chassis and in the vicinity of at least one of the openings; and
a targeted elastic material in at least a portion of the chassis, the targeted elastic material including at least one low stretch zone defining the fluid sealing gasket, and at least one high stretch zone spaced apart from and inward from the low stretch zone.

45. The pant-like garment of claim 44, further comprising a leg flap at least partially surrounding a leg opening, the leg flap including the targeted elastic material.

46. The pant-like garment of claim 44, further comprising a waist dam at least partially surrounding the waist opening, the waist dam including the targeted elastic material.

47. A method of producing a garment having a gasketed opening for a body part, comprising the steps of:
extruding a plurality of elastomeric first filaments from a plurality of spinning capillaries in at least one first spin plate region of a die;
extruding a plurality of elastomeric second filaments from a plurality of spinning capillaries in at least one second spin plate region of a die;
spacing the first filaments apart from the second filaments; and
joining the first and second filaments to a facing layer.

48. The method of claim 47, wherein the first and second filaments are spaced apart at a distance of at least 0.1 cm.

49. The method of claim 47, wherein the first and second filaments are spaced apart at a distance of at least 1.0 cm.

50. The method of claim 47, wherein the first and second filaments are spaced apart at a distance of at least 2.0 cm.

51. The method of claim 47, wherein the first and second filaments are spaced apart at a distance of at least 3.0 cm.

52. The method of claim 47, wherein the first spin plate region and the second spin plate region are in the same die.

53. The method of claim 47, wherein the first spin plate region and the second spin plate region are in separate dies.

54. The method of claim 47, wherein the first filaments are stretched before being joined to the facing layer.

55. The method of claim 47, wherein the first and second filaments are stretched before being joined to the facing layer.

56. The method of claim 47, further comprising the step of consolidating the first and second filaments with a second layer.

57. The method of claim 47, further comprising the step of inserting a barrier layer between the first filaments and the facing layer.

58. The method of claim 47, further comprising the step of inserting a barrier layer between at least two of the first filaments.
